(19) United States
(12) Patent Application Publication
(10) Pub. No.: US 2019/0223707 A1
(43) Pub. Date: Jul. 25, 2019

(54) CLEANING TOOL FOR ENDOSCOPE

(71) Applicant: OLYMPUS CORPORATION, Tokyo (JP)

(72) Inventor: Kohei FUJIWARA, Tokyo (JP)

(73) Assignee: OLYMPUS CORPORATION, Tokyo (JP)

(21) Appl. No.: 16/371,197

(22) Filed: Apr. 1, 2019

Related U.S. Application Data
(63) Continuation of application No. PCT/JP2017/029763, filed on Aug. 21, 2017.

(30) Foreign Application Priority Data
Oct. 11, 2016 (JP) 2016-200264

(51) Int. Cl. A61B 1/12; B08B 1/00; G02B 27/00

(52) U.S. Cl. CPC A61B 1/122; G02B 27/0006; B08B 1/002

(57) ABSTRACT

A cleaning tool for endoscope of the present invention includes a cylindrical main body which covers a bending operation member of an endoscope, and a cleaning member which is installed on an inner surface of the main body so as to clean the bending operation member.

### CROSS REFERENCE TO RELATED APPLICATION

This application is a continuation application of PCT/JP2017/029763 filed on Aug. 21, 2017 and claims benefit of Japanese Application No. 2016-200264 filed in Japan on Oct. 11, 2016, the entire contents of which are incorporated herein by this reference.

### BACKGROUND OF THE INVENTION

### 1. Field of the Invention

The invention relates to a cleaning tool for endoscope capable of easily performing cleaning of portions particularly having a complicated outer surface shape such as a bending operation member provided in an operation portion of an outer surface portion of an endoscope.

### 2. Description of the Related Art

Conventionally, endoscopes configured by having an insertion portion in an elongated tube shape have been widely used in a medical field, an industrial field and the like. Among the endoscopes, a medical endoscope that is used in the medical field is configured to be able to observe an organ and the like by inserting an insertion portion into a subject, for example, a body cavity of a living body, and to perform various kinds of treatments to the organ and the like in accordance with necessity by using a treatment instrument inserted into a treatment instrument insertion channel included in the endoscope. An industrial endoscope used in the industrial field is configured to be able to perform state observation, inspection and the like of a state in an object, for example, a scar, corrosion and the like by inserting an insertion portion into an object, for example, an apparatus, machine equipment or the like such as a jet engine and plant piping.

In this kind of endoscope, particularly in a medical endoscope, it is necessary to always maintain a clean state in use. Accordingly, when reprocessing (reuse) is considered, reliable cleaning treatment is required at each use.

However, a bending operation member or the like provided in an operation portion of an endoscope, for example, has a complicated outer shape, so that cleaning of the member or the like is performed by humans by using waste cloth or the like, and takes much time and effort.

As a contrivance for cleaning an object having a complicated outer shape, there has been conventionally seen a proposal by Japanese Patent Application Laid-Open Publication No. 2004-262378.

The cleaning tool disclosed by Japanese Patent Application Laid-Open Publication No. 2004-262378 described above or the like is a cleaning tool intended for wheels for vehicle or the like, a wheel for vehicle in a shape with depth and having a complicated design shape as an object. The cleaning tool is formed by adapting a cleaning member having elasticity in a shape approximated to a whole or a part of a surface of the wheel for vehicle to a design shape of a wheel surface. By causing the cleaning tool to act on the wheel surface mainly in a circular arc direction or a longitudinal direction, even a recessed site and small portions in complicated shapes in a wheel for vehicle with depth are cleaned more easily.

### SUMMARY OF THE INVENTION

A cleaning tool for endoscope of one aspect of the present invention includes a cylindrical main body which covers a bending operation member of an endoscope, and a cleaning member which is installed on an inner surface of the main body so as to clean the bending operation member.

### BRIEF DESCRIPTION OF THE DRAWINGS

FIG. 1 is a schematic perspective view illustrating a cleaning tool for endoscope of one embodiment of the present invention, and a part of an endoscope that is cleaned by using the cleaning tool for endoscope;

FIG. 2 is a vertical sectional view (a section taken along line [2]-[2] in FIG. 3) of the cleaning tool for endoscope of one embodiment of the present invention;

FIG. 3 is a plan view (plan view seen in a direction of an arrow reference sign [3] in FIG. 1) of the cleaning tool for endoscope of one embodiment of the present invention;

FIG. 4 is a vertical sectional view (a section taken along line [4]-[4] in FIG. 5) of a cleaning tool for endoscope of a first modification of one embodiment of the present invention;

FIG. 5 is a plan view of the cleaning tool for endoscope of the first modification in FIG. 4;

FIG. 6 is an operation explanatory diagram of the cleaning tool for endoscope of the first modification of one embodiment of the present invention;

FIG. 7 is a plan view illustrating a cleaning tool for endoscope of a second modification of one embodiment of the present invention;

FIG. 8 is a vertical sectional view (a section along line [8]-[8] in FIG. 9) of a cleaning tool for endoscope of a third modification of one embodiment of the present invention;

FIG. 9 is a plan view of the cleaning tool for endoscope of the third modification in FIG. 8;

FIG. 10 is a vertical sectional view (a section along line [10]-[10] in FIG. 11) of a cleaning tool for endoscope of a fourth modification of one embodiment of the present invention;

FIG. 11 is a plan view of the cleaning tool for endoscope of the fourth modification in FIG. 10;

FIG. 12 is a vertical sectional view (a section along line [12]-[12] in FIG. 13) of a cleaning tool for endoscope of a fifth modification of one embodiment of the present invention; and

FIG. 13 is a plan view of the cleaning tool for endoscope of the fifth modification in FIG. 12.

### DETAILED DESCRIPTION OF THE PREFERRED EMBODIMENT

Hereinafter, the present invention will be described according to an illustrated embodiment. The respective drawings for use in the following explanation are schematic illustrations. In order to show respective components with such sizes as to be recognizable on the drawings, the respective components may be shown by making the dimensional relationships, scales and the like of the respective components different for each of the components. Accordingly, the present invention is not limited to only illustrated modes, concerning quantities of the respective components, shapes of the respective components, ratios of the sizes of the respective components, relative positional relationships of the respective components and the like written in the respective drawings.

### Embodiment

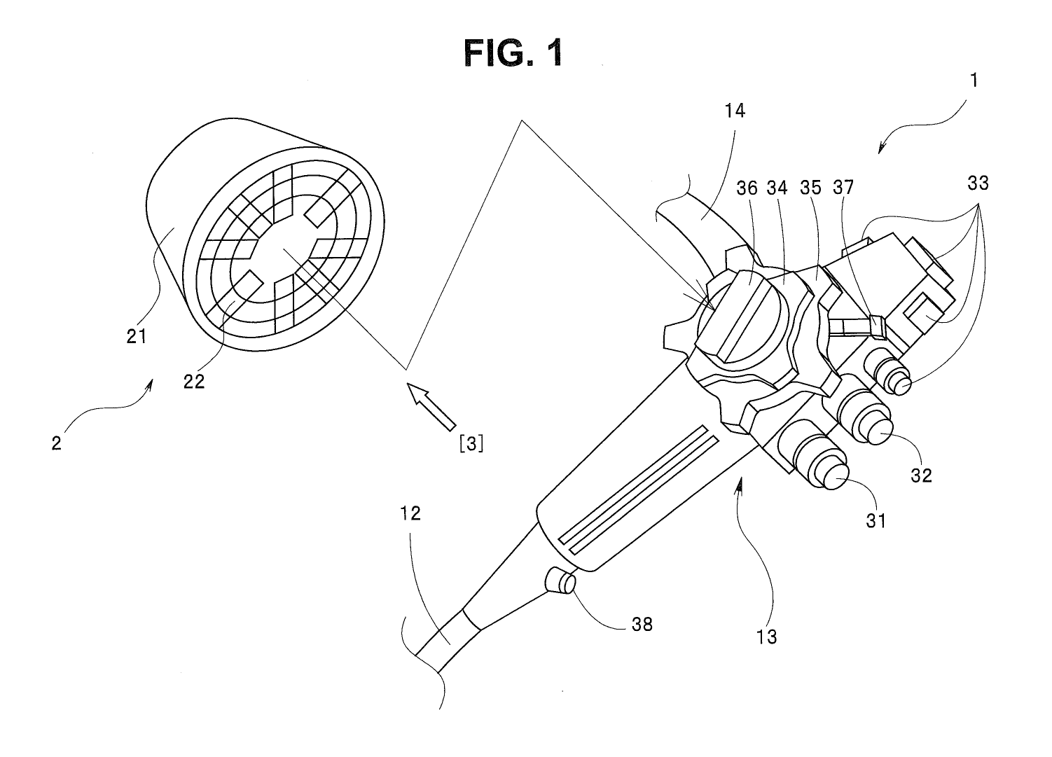

FIG. 1 is a schematic perspective view illustrating a cleaning tool for endoscope of one embodiment of the present invention, and a part of an endoscope that is cleaned by using the cleaning tool for endoscope.

First, a schematic configuration of an endoscope to be an object to be cleaned by using the cleaning tool for endoscope of the present embodiment will be described below by using FIG. 1.

A basic configuration of the endoscope which is an object to be cleaned by using the cleaning tool for endoscope of the present embodiment is substantially the same as an endoscope of a conventional general mode. Accordingly, in the following explanation, only outlines of individual components in an endoscope 1 will be described.

### One Embodiment (FIG. 1 to FIG. 3)

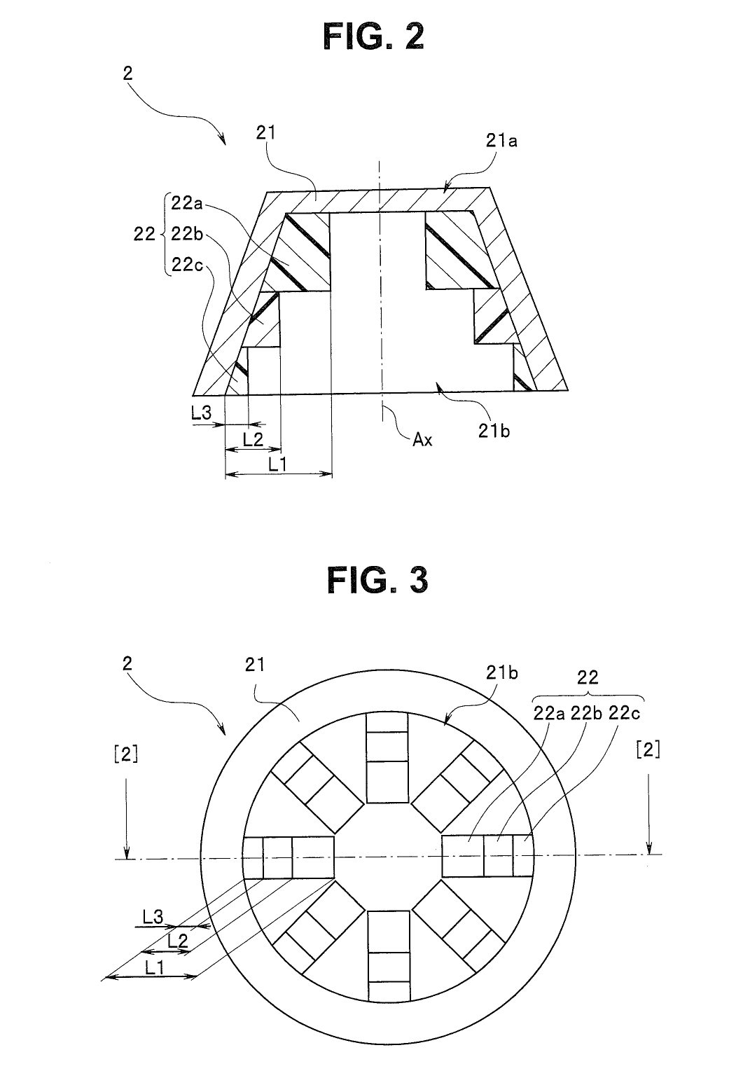

Further, a target part in the endoscope that is cleaned by the cleaning tool for endoscope of the present embodiment is particularly a bending operation member provided on an outer surface of an operation portion, and a part in a vicinity of the bending operation member. Accordingly, in FIG. 1, only components in the vicinity of the operation portion of the endoscope are illustrated. In the following explanation, only the components in a part in the vicinity of the operation portion of the endoscope will be mainly described. Note that component parts not illustrated are parts that are not directly related to the present invention, so that explanation of the parts will also be omitted. In FIG. 1, only a schematic configuration of the cleaning tool for endoscope of the present embodiment is illustrated. Note that a detailed configuration of the cleaning tool for endoscope of the present embodiment will be described later by using FIGS. 2 and 3.

As illustrated in FIG. 1, the endoscope 1 is mainly configured by including an insertion portion 12 configured to be inserted into a body cavity of a subject, an operation portion 13 connectively provided at a proximal end side in a long axis direction (insertion direction) of the insertion portion 12, a universal cord 14 that is extended from the operation portion 13 and the like.

The above described insertion portion 12 is a tubular member that has a distal end and a proximal end, is formed into an elongated tubular shape having flexibility, and is configured to be inserted into a body cavity from a distal end side along a longitudinal axis. The insertion portion 12 is configured by connecting a distal end portion (not illustrated), a bending portion (not illustrated) and a flexible tube portion (not illustrated) in order from a distal end side. An inside of the insertion portion 12 allows insertion of a bending cable or the like that performs a bending operation of the above described bending portion (not illustrated), besides a plurality of conduits (a treatment instrument insertion channel, an air feeding and water feeding conduit, a suction conduit and the like), a light guide, signal cables and the like.

The above described operation portion 13 is a component part that is connected to a proximal end of the above described insertion portion 12, includes a plurality of operation members and the like for receiving operation inputs by an operator on an outer surface, and allows insertion and disposition of the plurality of conduits (the treatment instrument insertion channel, the air feeding and water feeding conduit, the suction conduit and the like), the light guide, the signal cables, the bending cable and the like that are extended from the above described insertion portion 12 in an inside, and further includes a bending mechanism or the like for realizing the bending operation of the above described bending portion (not illustrated). The outer surface of the operation portion 13 is configured to also serve as a grasping portion for a user to grasp when the user uses the endo scope 1.

A plurality of operation members that are provided on the outer surface of the above described operation portion 13 include, for example, an air feeding and water feeding button 31, a suction button 32, a plurality of remote switches 33, a plurality of bending operation members (34, 35, 36, 37) and the like.

The air feeding and water feeding button 31 and the suction button 32 are push-type operation members for performing an air feeding and water feeding operation and a suction operation to an inside of a body cavity of a subject via the insertion portion 12 (that allows insertion of the air feeding and water feeding conduit and the suction conduit).

A plurality of remote switches 33 are push-type operation members for performing image control instructions of a freeze (image stop) operation, a release (still image recording) operation and the like, a switching operation of a light measurement method, a print execution operation, a switching operation to an enlarged display mode, a start/stop operation of video recording and the like.

The plurality of bending operation members include a lateral bending operation knob 34, a vertical bending operation knob 35, a lateral bending fixing knob 36, and a vertical bending fixing lever 37. The four operation members are configured to be rotatable forward and reversely on a same axis.

Among the bending operation members, the lateral bending operation knob 34 and the vertical bending operation knob 35 are rotary operation members for the bending portion (not illustrated) to bend around an insertion axis in a left-and-right (LR) direction and an up-and-down (UD) direction respectively by being rotationally operated.

The above described bending fixing knob 36 is a rotary operation member for fixing the lateral bending operation knob 34 in a desired position when the lateral bending operation knob 34 is operated. The above described bending fixing lever 37 is a rod-shaped operation member for fixing the vertical operation knob 35 in a desired position when the vertical bending operation knob 35 is operated.

Accordingly, a bending portion (not illustrated) of the above described insertion portion 12 is configured to be able to bend in four directions, that are up, down, left and right, by rotationally operating the lateral bending operation knob 34 and the vertical bending operation knob 35 separately properly.

A treatment instrument insertion opening 38 is provided in a part close to the distal end of the above described operation portion 13. A proximal end of the treatment instrument insertion channel (not illustrated) that is inserted through the inside of the above described insertion portion 12 from a distal end to the proximal end side to reach the inside of the operation portion 13 is connected to the treatment instrument insertion opening 38.

The universal cord 14 is a cord that has a proximal end extended from a side portion of the operation portion 13, includes, at a distal end, an endoscope connector (not illustrated) that is detachably connected to a light source apparatus (not illustrated), and is formed to have flexibility. An inside of the universal cord 14 allows insertion of the plurality of conduits (the air feeding and water feeding conduit, the suction conduit and the like) that are inserted through the inside of the above described operation portion 13 from the above described insertion portion 12, the light guide, the signal cables and the like. A configuration of the part in the vicinity of the operation portion 13 in the above described endoscope 1 is as described above.

The cleaning tool 2 for endoscope of the present embodiment is a cleaning tool for cleaning the plurality of bending operation members (34, 35, 36, 37) provided in the operation portion 13 in the endoscope 1 configured as described above. Note that in the example shown in the present embodiment, the bending fixing lever 37 is in a form such that a part of the bending fixing lever 37 can be cleaned.

Next, a detailed configuration of the cleaning tool 2 for endoscope of the present embodiment will be described below. FIG. 2 is a vertical sectional view of the cleaning tool for endoscope of the present embodiment. FIG. 2 illustrates a section along line [2]-[2] in FIG. 3. FIG. 3 is a plan view of the cleaning tool for endoscope of the present embodiment. FIG. 3 illustrates a plane as viewed in a direction of an arrow reference sign [3] in FIG. 1.

As illustrated in FIG. 1 to FIG. 3, the cleaning tool 2 for endoscope of the present embodiment is mainly configured by a main body 21 and a cleaning member 22.

The main body 21 is a cylindrical hollow member that is formed in a size capable of being externally fitted on the lateral bending operation knob 34, the vertical bending operation knob 35 and the lateral bending fixing knob 36, out of the plurality of bending operation members provided in the endoscope 1. As a material forming the main body 21, a resin molded product or the like is used, for example

The main body 21 illustrated in the present embodiment is specifically such that one end surface in a direction along a central axis Ax (refer to FIG. 2) of a substantially cylindrical shape is formed by being closed, for example, and the other end surface is formed by being opened. Here, the above described one end surface in the main body 21 is referred to as a closed surface 21a. An opening on the above described other end surface is designated by reference sign 21b (refer to FIG. 2).

An outside diameter of the other end surface where the opening 21b of the main body 21 is formed is formed to be larger than an outside diameter of the closed surface 21a. That is to say, the main body 21 is formed into a tapered shape toward the one end surface where the closed surface 21a is formed from the other end surface where the opening 21b is formed.

The above described opening 21b is formed into such a size as to be externally fittable onto the lateral bending operation knob 34, the vertical bending operation knob 35 and the lateral bending fixing knob 36. In this case, the opening 21b is formed to also cover a part of the bending fixing lever 37.

The cleaning member 22 is disposed to protrude inward in a radial direction from an inner circumferential surface of the main body 21. A plurality of the above described cleaning members 22 illustrated in the present embodiment are arranged around the central axis Ax of the above described main body 21 (refer to FIG. 3).

Further, a plurality of the above described cleaning members 22 are arranged in parallel in a direction of the central axis Ax of the above described main body 21 (refer to reference signs 22a, 22b and 22c in FIG. 2 and FIG. 3). In this case, among the plurality of cleaning members 22 which are arranged in parallel in the direction of the central axis Ax, the cleaning members are referred to as a cleaning member (A) 22a, a cleaning member (B) 22b and a cleaning member (C) 22c in order from a side close to the closed surface 21a. The cleaning member (A) 22a, the cleaning member (B) 22b and the cleaning member (C) 22c are formed such that protruding amounts by which the cleaning member (A) 22a, the cleaning member (B) 22b and the cleaning member (C) 22c protrude inward in the radial direction from the inner circumferential surface of the main body 21, are different. More specifically, as illustrated in FIG. 2 and FIG. 3, for example, the cleaning member (A) 22a, the cleaning member (B) 22b and the cleaning member (C) 22c are formed so as to satisfy L1>L2>L3, where a protruding amount by which the cleaning member (A) 22a protrudes inward is L1, a protruding amount by which the cleaning member (B) 22b protrudes inward is L2, and a protruding amount by which the cleaning member (C) 22c protrudes inward is L3.

The reason why the cleaning member (A) 22a, the cleaning member (B) 22b and the cleaning member (C) 22c are configured in this way is that the cleaning member (A) 22a, the cleaning member (B) 22b and the cleaning member (C) 22c are adapted to shapes of target parts to be cleaned by using the cleaning tool 2 for endoscope, that is to say the lateral bending operation knob 34, the vertical bending operation knob 35, and the lateral bending fixing knob 36 of the endoscope 1. By configuring the cleaning member (A) 22a, the cleaning member (B) 22b and the cleaning member (C) 22c in this way, the cleaning member (A) 22a is in a shape suitable for mainly cleaning the lateral bending fixing knob 36, the cleaning member (B) 22b is in a shape suitable for mainly cleaning the lateral bending operation knob 34, and the cleaning member (C) 22c is in a shape suitable for mainly cleaning the vertical bending operation knob 35.

As the above described cleaning member 22, a member formed from a stretchable and soft material having flexibility, for example, a sponge or the like formed by using a synthetic resin such as polyurethane is applied.

### First Modification

Note that a mode may be adopted, in which a second cleaning member in a different shape is further provided on an inner surface of the closed one end surface (closed surface 21a) of the main body 21 in addition to the configuration of the cleaning tool 2 for endoscope of the above described one embodiment.

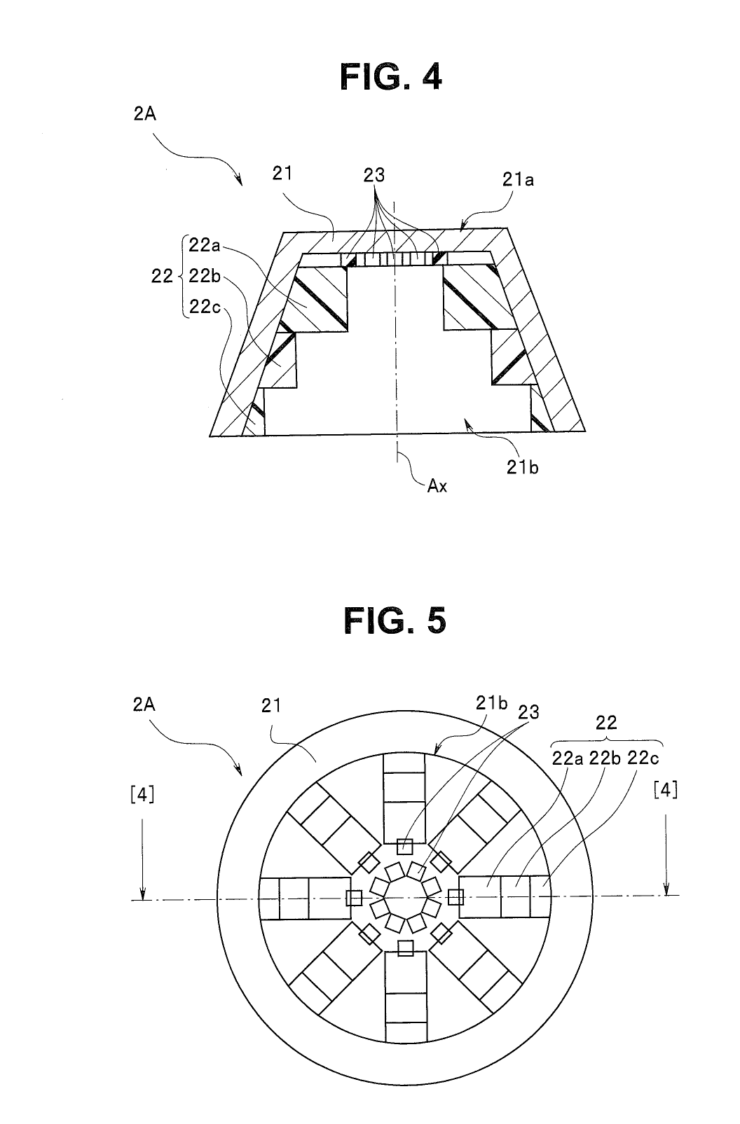

FIG. 4 and FIG. 5 are views each illustrating a cleaning tool for endoscope of a first modification of the above described embodiment. Of the drawings, FIG. 4 is a vertical sectional view (a section along line [4]-[4] in FIG. 5) corresponding to FIG. 2 of the above described embodiment. FIG. 5 is a plan view corresponding to FIG. 3 in the above described one embodiment.

A cleaning tool 2A for endoscope of the first modification differs only in that a second cleaning member 23 is provided in addition to the configuration of the cleaning tool 2 for endoscope of the aforementioned first embodiment.

The above described second cleaning member 23 is provided on the inner surface of the closed one end surface (closed surface 21a) of the main body 21, and is disposed to protrude toward the other end (that is to say, the opening 21b) from the inner surface of the closed surface 21a. The above described second cleaning member 23 illustrated in the present modification is configured by providing a plurality of cleaning members aligned in a circumferential direction around the central axis Ax of the above described closed surface 21a in two rows in the radial direction (refer to FIG. 5).

As for the above described second cleaning member 23, a sponge or the like formed by using a synthetic resin such as polyurethane is also applied similarly to the above described cleaning member 22.

The lateral bending operation knob 34, the vertical bending operation knob 35 and the lateral bending fixing knob 36 of the endoscope 1 which are the objects to be cleaned are cleaned by using the cleaning tool 2A for endoscope illustrated in the first modification example of the above described one embodiment configured in this way. An operation at a time of the cleaning will be briefly described below.

Note that the cleaning that is performed by using the cleaning tool 2A for endoscope also includes spot cleaning or the like for a predetermined part at the time of cleaning the endoscope manually in addition to preliminary cleaning before mechanical cleaning that is performed in a full scale by using a cleaner, for example.

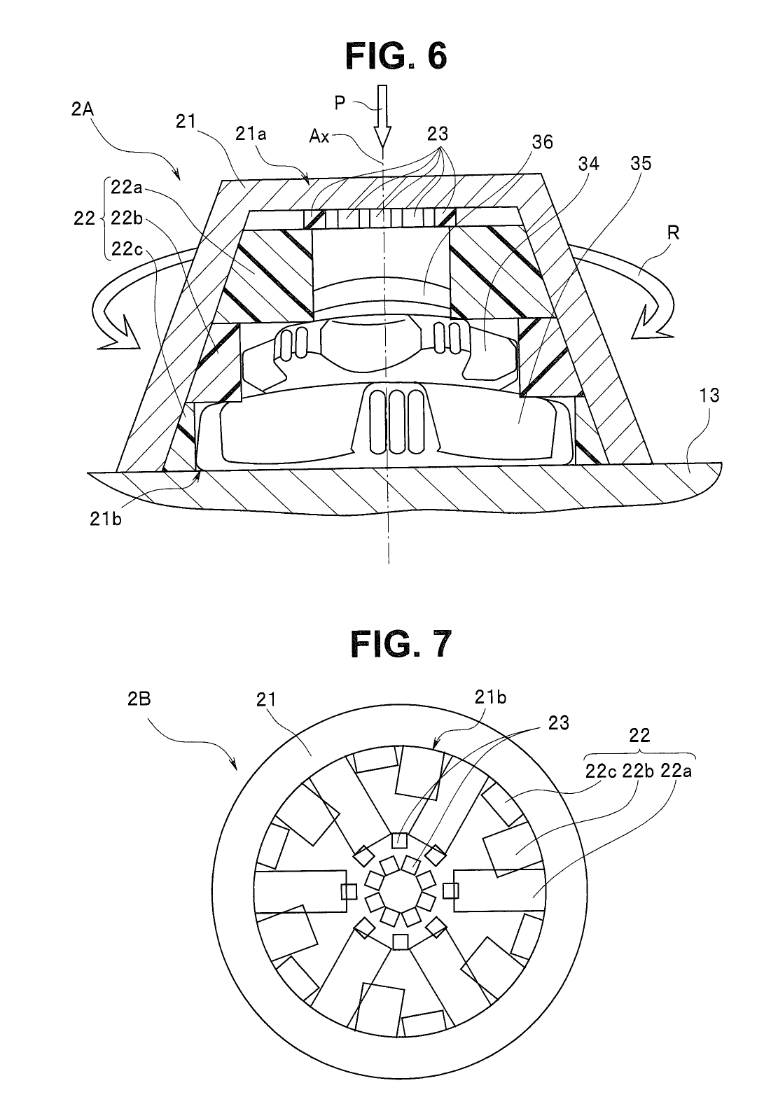

FIG. 6 is a view explaining an operation of the cleaning tool for endoscope of the first modification of the above described one embodiment. Note that FIG. 6 is a vertical sectional view corresponding to FIG. 4 of the first modification.

First, a predetermined cleaning agent or the like is attached in advance to the cleaning member 22 of the cleaning tool 2A for endoscope.

Next, the user holds the outer circumferential surface of the main body 21 in his or her palm or the like, and externally fits the opening 21b of the main body 21 onto a predetermined part of the endoscope 1. Thereby, the main body 21 is placed to cover the lateral bending operation knob 34, the vertical bending operation knob 35 and the lateral bending fixing knob 36 as illustrated in FIG. 6. At this time, in the main body 21, the cleaning member 22 is brought into a state where the cleaning member 22 is properly compressed by outer surfaces of the above described respective knobs (34, 35, 36). In this state, the user performs forward and reverse rotational operation in a direction of an arrow reference sign R in FIG. 6 in a state where the user grasps the main body 21 from the outer surface. Thereby, the cleaning member 22 deforms in accordance with the shapes of the outer surfaces while being rubbed against the outer surfaces of the respective knobs (34, 35, 36), moves in a circumferential direction along the outer surfaces and cleans the outer surfaces.

When the above described rotational operation is performed, if a pressing force in a direction of an arrow reference sign P in FIG. 6 is simultaneously applied to the main body 21, cleaning of the surface parallel with the closed surface 21a of the outer surfaces of the respective knobs (34, 35, 36) can be effectively performed, and the second cleaning member 23 can be used.

Note that the cleaning tool 2 for endoscope of the present embodiment may be of a disposable type that is directly discarded after a single use, or may be of a reusable type.

As described above, according to the above described embodiment and the above described first modification, cleaning of the predetermined parts can also be performed by only performing a simple operation to parts in complicated shape by preparing the exclusive cleaning tool 2 for endoscope in the form adapted to a shape of a vicinity of the bending operation knob in the endoscope 1.

The cleaning tool 2 for endoscope has a simple configuration, and can be produced at extremely low cost by using an ordinary material (for example, a resin molded product or the like for the main body, a sponge or the like for the cleaning members), so that the cleaning tool 2 for endoscope is efficient when used as the cleaning tool for endoscope of a disposal type.

### Second Modification

Note that the configuration of the cleaning tool for endoscope of the present invention is not limited to the modes illustrated by the aforementioned one embodiment and first modification of the aforementioned one embodiment.

For example, FIG. 7 is a plan view illustrating a cleaning tool for endoscope of a second modification of the above described one embodiment. FIG. 7 is a plane view corresponding to FIG. 3 of the above described embodiment.

In a cleaning tool 2B for endoscope of a second modification illustrated in FIG. 7, disposition of the cleaning member (A) 22a, the cleaning member (B) 22b and the cleaning member (C) 22c in the cleaning member 22 is configured to be different from the disposition in the above described one embodiment (and the first modification).

That is to say, the second modification is the same as the above described one embodiment (and the first modification) in that the cleaning members 22 are disposed to protrude inward in the radial direction from the inner circumferential surface of the main body 21.

In the present modification, pluralities of cleaning members (A) 22a, cleaning members (B) 22b and the cleaning member (C) 22c are arranged around the central axis Ax of the above described main body 21. The respective cleaning members (A) 22a, cleaning members (B) 22b and cleaning member (C) 22c are configured to be disposed in positions shifted in the radial direction so as not to overlap one another in the direction of the central axis Ax.

More specifically, for example, as illustrated in FIG. 7, pluralities of cleaning members (A) 22a, cleaning members (B) 22b and cleaning members (C) 22c are arranged around the central axis Ax of the above described main body 21. The cleaning members (B) 22b are disposed by being shifted in the radial direction with respect to disposition of the cleaning members (A) 22a so as not to overlap the cleaning members (A) 22a in the direction of the central axis Ax. Likewise, the cleaning members (C) 22c are disposed by being shifted in the radial direction with respect to disposition of the cleaning members (B) 22b so as not to overlap the cleaning members (B) 22b in the direction of the central axis Ax.

The other configurations are substantially the same as the above described first modification. The second modification also illustrates the example in which a sponge is applied, for example, as the cleaning member 22.

According to the above described second modification configured in this way, similar effects to the effects of the aforementioned one embodiment and first modification also can be obtained.

### Third Modification

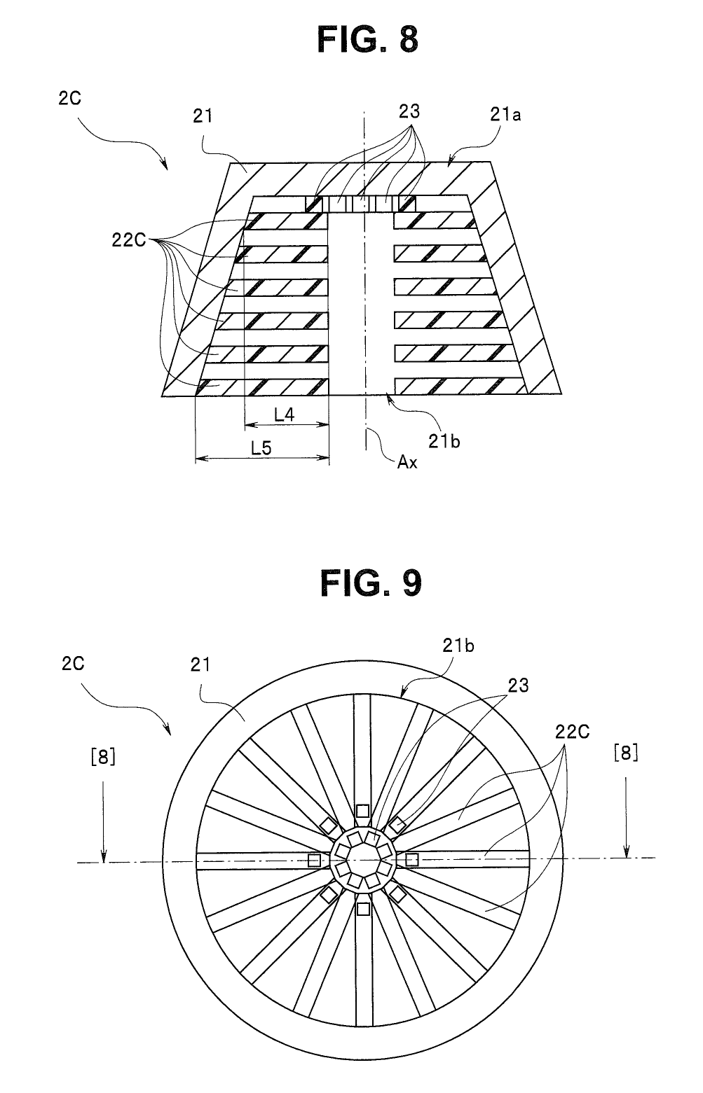

FIG. 8 and FIG. 9 are, for example, views each illustrating a cleaning tool for endoscope of a third modification of the above described one embodiment. Of the drawings, FIG. 8 is a vertical sectional view (a section along line [8]-[8] in FIG. 9) corresponding to FIG. 2 of the above described one embodiment. FIG. 9 is a plan view corresponding to FIG. 3 in the above described one embodiment.

In a cleaning tool 2C for endoscope of the third modification, a shape of a cleaning member 22C is configured to be different.

That is to say, the cleaning member 22C of the third modification is the same as the above described one embodiment and the above described respective modifications in that the cleaning member 22C is disposed to protrude inward in the radial direction from the inner circumferential surface of the main body 21.

In the present modification, the cleaning member 22C differs in that a plurality of cleaning members protruding inward in the radial direction are each formed into an elongated shape to have more flexibility and to be softer as compared with the cleaning members illustrated in the aforementioned one embodiment and respective modifications.

Individual cleaning members configuring the above described cleaning member 22C are provided to protrude in the radial direction inward from the inner circumferential surface of the main body 21. In this case, the plurality of cleaning members are provided at predetermined intervals in the circumferential direction, and are provided in parallel at predetermined intervals in the axial direction.

Note that the cleaning member 22C of the present modification is formed to satisfy L4<L5, as illustrated in FIG. 8, when a protruding amount inward in the radial direction from the inner circumferential surface of the main body 21 of the cleaning member 22C which is arranged close to the closed surface 21a is L4, and a protruding amount inward in the radial direction from the inner circumferential surface of the main body 21 of the cleaning member 22C that is placed close to the opening 21b is L5. That is to say, distances to the central axis Ax of the main body 21 from respective distal end portions of the plurality of cleaning members configuring the cleaning member 22C are all formed to be substantially equal.

The other configurations are substantially the same as the above described first modification. In the third modification, an example where a sponge is applied as the cleaning member 22C is illustrated.

By the above described third modification configured in this way, similar effects to the effects of the aforementioned one embodiment and first modification can be obtained.

Note that though not illustrated, a modification is also conceivable, which is configured such that the respective plurality of cleaning members configuring the above described cleaning member 22C are disposed in positions shifted in the radial direction so as not to overlap one another in the direction of the central axis Ax.

### Fourth Modification

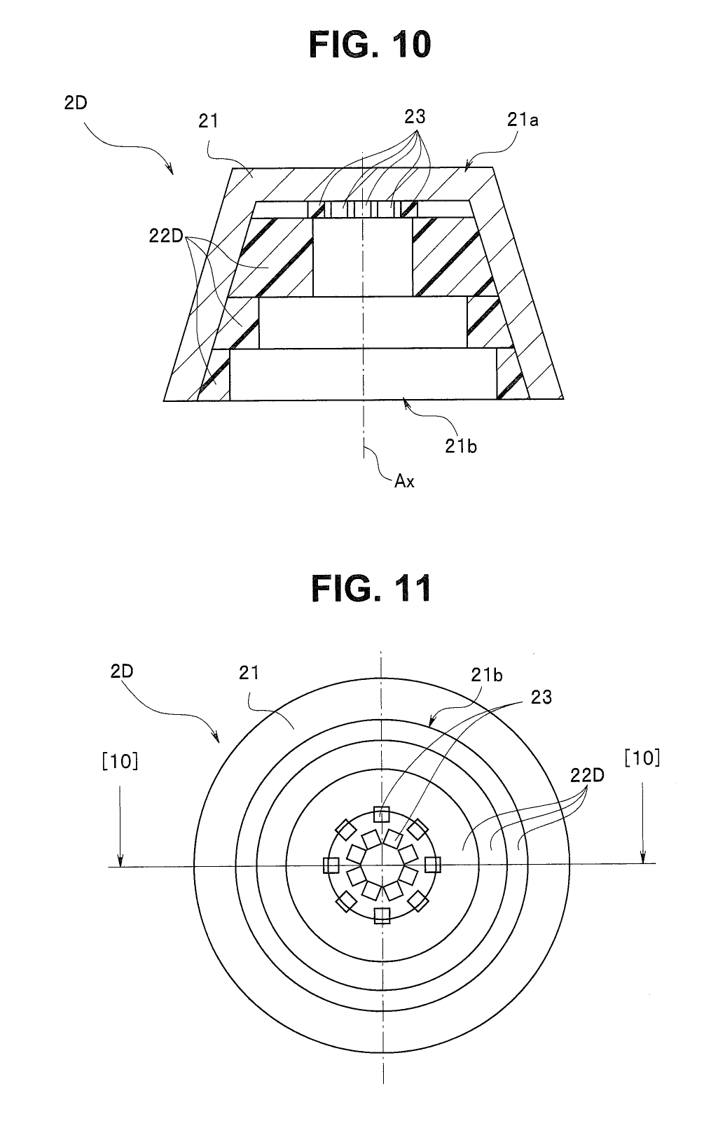

FIG. 10 and FIG. 11 are, for example, views each illustrating a cleaning tool for endoscope of a fourth modification of the above described one embodiment. Of these drawings, FIG. 10 is a vertical sectional view corresponding to FIG. 2 of the above described one embodiment (a section along line [10]-[10] in FIG. 11). FIG. 11 is a plan view corresponding to FIG. 3 in the above described one embodiment.

A cleaning tool 2D for endoscope of the fourth modification differs in that a cleaning member 22D is configured by a sponge or the like formed into a ring shape.

That is to say, the cleaning member 22D of the fourth modification is formed to protrude inward in the radial direction from the inner circumferential surface of the main body 21 and into a ring shape in a faun continuing in the circumferential direction.

The other configurations are substantially the same as the above described first modification. The fourth modification also illustrates an example in which a sponge, for example, is applied as the cleaning member 22D.

By the above described fourth modification configured in this way, similar effects to the effects of the aforementioned one embodiment and first modification can also be obtained.

### Fifth Modification

The aforementioned one embodiment and respective modifications each illustrates the example in which a sponge is applied as the cleaning member, but the cleaning member is not limited to this. For example, as the cleaning member, a plurality of brushes that are formed by densely implanting a plurality of fibrous materials can also be applied.

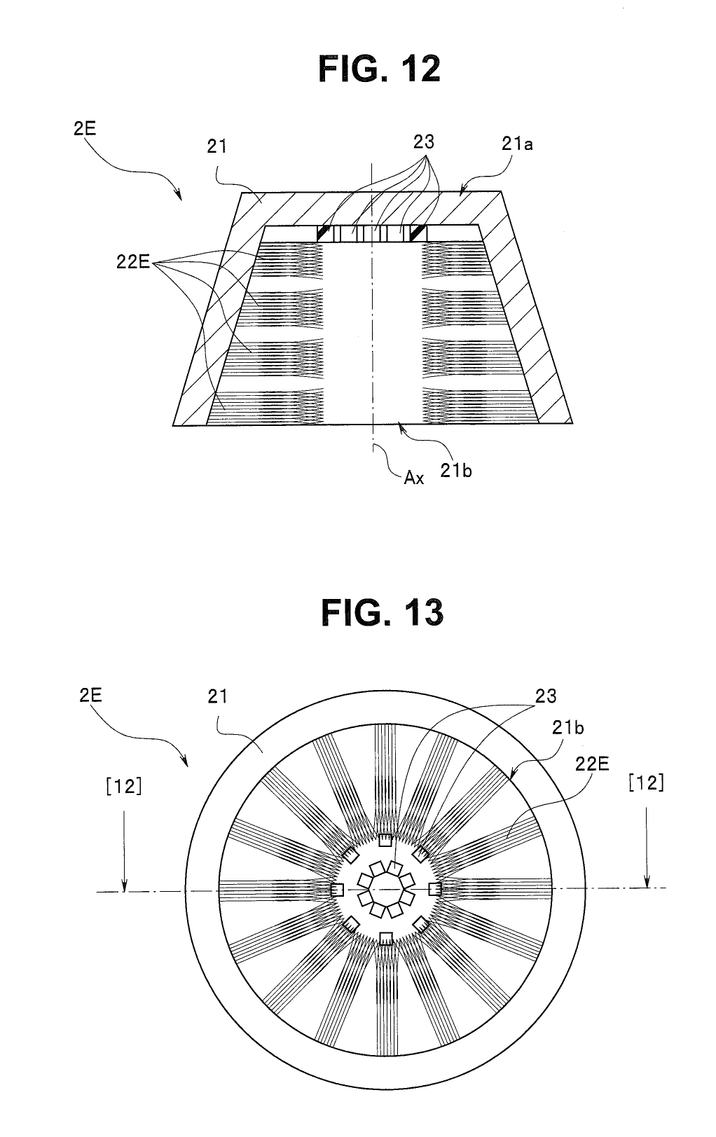

FIG. 12 and FIG. 13 are views illustrating a cleaning tool for endoscope of a fifth modification of the above described one embodiment. Of the drawings, FIG. 12 is a vertical sectional view (a section along line [12]-[12] in FIG. 13) corresponding to FIG. 2 of the above described one embodiment. FIG. 13 is a plan view corresponding to FIG. 3 of the above described one embodiment.

A cleaning tool 2E for endoscope of the present modification illustrates an example in which a cleaning member 22E that is formed of a plurality of brushes is configured in substantially the same form as the aforementioned third modification (FIG. 8, FIG. 9), in place of the cleaning member 22C formed of a sponge of the aforementioned third modification (FIG. 8, FIG. 9).

The cleaning member 22E in the present modification is formed by providing a plurality of brushes in a form in which a plurality of fibrous materials are densely implanted to protrude inward in the radial direction from the inner circumferential surface of the main body 21.

That is to say, in the cleaning tool 2E for endoscope of the present modification, the above described cleaning member 22E is configured by arranging a plurality of brush bundles in parallel at predetermined intervals in the circumferential direction, and by also arranging the plurality of brush bundles in parallel at predetermined intervals in the axial direction as well. The brush bundle is formed by densely implanting a predetermined number of fibrous materials and bundling the predetermined number of fibrous materials as one bundle. The other configurations are substantially the same as the above described first modification.

According to the above described fifth modification configured as above, a similar effect to the effects of the aforementioned one embodiment and first modification can also be obtained.

Needless to say, the present invention is not limited to the aforementioned embodiment, but various modifications and applications can be carried out within the range without departing from the gist of the invention. Further, the above described embodiment includes the inventions in various stages, and various inventions can be extracted by an appropriate combination in a plurality of components that are disclosed. For example, even when some components are deleted from all the components shown in the above described one embodiment the configuration from which the components are deleted can be extracted as the invention if the problem to be solved by the invention can be solved and the effect of the invention can be obtained. Furthermore, components across different embodiments may be properly combined. The invention is not restricted by a specific embodiment of the invention except that the invention is limited by the accompanying claims.

The present invention is applicable to not only an endoscope control apparatus in the medical field but also an endoscope control apparatus in the industrial field.

What is claimed is:

1. A cleaning tool for endoscope comprising:
a cylindrical main body which covers a bending operation member of an endoscope; and
a cleaning member which is installed on an inner surface of the main body so as to clean the bending operation member.

2. The cleaning tool for endoscope according to claim 1,
wherein the cleaning member in plurality is arranged around a central axis of the main body.

3. The cleaning tool for endoscope according to claim 1,
wherein the cleaning member in plurality is arranged in parallel in an axial direction of the main body.

4. The cleaning tool for endoscope according to claim 1,
wherein the cleaning member is formed by a plurality of brushes.

5. The cleaning tool for endoscope according to claim 1,
wherein the cleaning member is formed by a plurality of sponges.

6. The cleaning tool for endoscope according to claim 1,
wherein the cleaning member protrudes in the radial direction of the main body.

7. The cleaning tool for endoscope according to claim 1,
wherein the cleaning member is a sponge formed into a ring shape.

8. The cleaning tool for endoscope according to claim 1,
wherein the main body has one end surface closed in an axial direction.

9. The cleaning tool for endoscope according to claim 8, further comprising:
a second cleaning member disposed on an inner surface of the closed one end surface in the main body, to protrude toward another end.

10. The cleaning tool for endoscope according to claim 3,
wherein the cleaning member in plurality is formed such that protruding amounts by which the cleaning member in plurality protrudes inward in the radial direction from the inner circumferential surface of the main body are different respectively.

11. The cleaning tool for endoscope according to claim 8,
wherein the main body has another end surface in the axial direction formed to be opened, and an outside diameter of the other end surface formed to be larger than an outside diameter of the one end surface.

12. The cleaning tool for endoscope according to claim 1,
wherein the cleaning member protrudes inward in a radial direction from an inner circumferential surface of the main body.
